(19) United States
(12) Patent Application Publication
(10) Pub. No.: US 2025/0109371 A1
(43) Pub. Date: Apr. 3, 2025

(54) DEVICES, SYSTEMS, AND METHODS FOR ORGANOID ANALYSIS

(71) Applicant: Diagnostic Biochips, Inc., Glen Burnie, MD (US)

(72) Inventors: Brian Glenn JAMIESON, Severna Park, MD (US); Fan WU, Silver Spring, MD (US); Dongyoul LEE, Laurel, MD (US); Andrew S. DIRKS, Annapolis, MD (US); Nicholas Talbot WATKINS, Severna Park, MD (US)

(73) Assignee: Diagnostic Biochips, Inc, Glen Burnie, MD (US)

(21) Appl. No.: 18/899,388

(22) Filed: Sep. 27, 2024

Related U.S. Application Data
(60) Provisional application No. 63/586,770, filed on Sep. 29, 2023.

(51) Int. Cl. C12M 1/36; C12M 1/32

(52) U.S. Cl. CPC C12M 41/48; C12M 23/12

(57) ABSTRACT

Disclosed are systems and methods for organoid analysis, the device including a reader head having a plurality of probes, wherein each of the plurality of probes includes a shank with a plurality of recording sites, and a plate having a plurality of wells, wherein each well of the plurality of wells is configured to receive at least one organoid, wherein the reader head is configured to automatically probe the at least one organoid and to measure a property of the at least one organoid or to stimulate electrically active cells using the plurality of recording sites of the plurality of probes.

### CROSS-REFERENCE TO RELATED APPLICATION(S)

This application claims the benefit of pending U.S. Provisional Patent Application No. 63/586,770, filed on Sep. 29, 2023, the entirety of which is incorporated herein by reference.

### TECHNICAL FIELD

Various embodiments of the present disclosure pertain generally to devices, systems, and methods for organoid probing and analyzing data from organoid probing. More specifically, particular embodiments of the present disclosure relate to devices, systems, and methods for measuring organoid electrical activity using an organoid recording device.

### BACKGROUND

Organoids are three-dimensional, differentiated cell structures that are cultured from induced pluripotent stem cells (iPSCs) and can be used as models of specific organs such as the brain or heart. Organoids are utilized to, for example, develop and discover drugs or other therapies and/or medical techniques. The background description provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY OF THE DISCLOSURE

According to certain aspects of the disclosure, devices, systems, and methods are disclosed for organoid probing and analysis of the probing.

In one aspect, a device for organoid analysis is disclosed. The device may including a reader head having a plurality of probes, wherein each of the plurality of probes includes a shank with a plurality of recording sites, and a plate having a plurality of wells, wherein each well of the plurality of wells is configured to receive at least one organoid, wherein the reader head is configured to automatically probe the at least one organoid and to measure a property of the at least one organoid or to stimulate electrically active cells using the plurality of recording sites of the plurality of probes.

In another aspect, a device for organoid analysis is disclosed. The device may include a plurality of probes, wherein each of the plurality of probes includes a shank with a plurality of recording sites, a camera, and a plate having a plurality of wells, wherein each well of the plurality of wells is configured to receive at least one organoid, and wherein the plate includes at least one fiducial, wherein the camera is configured to interpret the at least one fiducial, and wherein the plurality of probes aligned with a well of the plurality of wells based on interpretation of the at least one fiducial.

In another aspect, a system for organoid analysis is disclosed. The system may include an organoid probing device having: at least one probe including a shank with a plurality of recording sites, and a plate having a plurality of wells, wherein each well of the plurality of wells is configured to receive at least one organoid, and a control and analysis unit configured to receive data from the plurality of recording sites and to automatically position the at least one probe based on the data.

It is to be understood that both the foregoing general description and the following detailed description are exemplary and explanatory only and are not restrictive of the disclosed embodiments, as claimed.

### BRIEF DESCRIPTION OF THE FIGURES

The accompanying drawings, which are incorporated in and constitute a part of this specification, illustrate various exemplary embodiments and together with the description, serve to explain the principles of the disclosed embodiments.

FIG. 1 depicts an exemplary organoid recording system, according to one or more embodiments.

FIG. 2 depicts an exemplary probe, according to one or more embodiments.

FIG. 3A depicts a further exemplary probe, according to one or more embodiments.

FIG. 3B depicts exemplary probes of FIG. 3A in an exemplary configuration, according to one or more embodiments.

FIG. 4A depicts an exemplary plate, according to one or more embodiments.

FIG. 4B depicts a cross-section of an exemplary well of FIG. 4A, according to one or more embodiments.

FIG. 5 depicts an exemplary environment for classifying an organoid, according to one or more embodiments.

FIG. 6 depicts an exemplary method for classifying an organoid, according to one or more embodiments.

FIG. 7 depicts an exemplary schematic for classifying an organoid, according to one or more embodiments.

FIG. 8 depicts an example machine learning training flow chart, according to some embodiments of the disclosure.

FIG. 9 depicts a simplified functional block diagram of a computer, according to one or more embodiments.

### DETAILED DESCRIPTION

The disclosure may be understood with reference to the following description and the appended drawings, wherein like elements are referred to with the same reference numerals.

The terminology used below may be interpreted in its broadest reasonable manner, even though it is being used in conjunction with a detailed description of certain specific examples of the present disclosure. Indeed, certain terms may even be emphasized below; however, any terminology intended to be interpreted in any restricted manner will be overtly and specifically defined as such in this Detailed Description section. Both the foregoing general description and the following detailed description are exemplary and explanatory only and are not restrictive of the features, as claimed.

In this disclosure, the term “based on” means “based at least in part on.” The singular forms “a,” “an,” and “the” include plural referents unless the context dictates otherwise. The term “exemplary” is used in the sense of “example” rather than “ideal.” The terms “comprises,” “comprising,” “includes,” “including,” or other variations thereof, are intended to cover a non-exclusive inclusion such that a process, method, or product that comprises a list of elements does not necessarily include only those elements, but may include other elements not expressly listed or inherent to such a process, method, article, or apparatus. The term “or” is used disjunctively, such that “at least one of A or B” includes, (A), (B), (A and A), (A and B), etc. Relative terms, such as, “substantially,” “approximately,” “about,” and “generally,” are used to indicate a possible variation of ±10% of a stated or understood value.

It will also be understood that, although the terms first, second, third, etc. are, in some instances, used herein to describe various elements, these elements should not be limited by these terms. These terms are only used to distinguish one element from another. For example, a first element could be termed a second element, and, similarly, a second element could be termed a first element, without departing from the scope of the various described embodiments. The first element and the second element are both contacts, but they are not the same contact.

As used herein, the term “if” is, optionally, construed to mean “when” or “upon” or “in response to determining” or “in response to detecting,” depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event],” depending on the context.

The systems, devices, and methods disclosed herein are described in detail by way of examples and with reference to the figures. The examples discussed herein are examples only and are provided to assist in the explanation of the apparatuses, devices, systems, and methods described herein. None of the features or components shown in the drawings or discussed below should be taken as mandatory for any specific implementation of any of these devices, systems, or methods unless specifically designated as mandatory.

Also, for any methods described, regardless of whether the method is described in conjunction with a flow diagram, it should be understood that unless otherwise specified or required by context, any explicit or implicit ordering of steps performed in the execution of a method does not imply that those steps must be performed in the order presented but instead may be performed in a different order or in parallel.

As used herein, the term “exemplary” is used in the sense of “example,” rather than “ideal.” Moreover, the terms “a” and “an” herein do not denote a limitation of quantity, but rather denote the presence of one or more of the referenced items.

As used herein, a “machine learning model” generally encompasses instructions, data, or a model configured to receive input, and apply one or more of a weight, bias, classification, or analysis on the input to generate an output. The output may include, for example, a classification of the input, an analysis based on the input, a design, process, prediction, or recommendation associated with the input, or any other suitable type of output. A machine learning model is generally trained using training data, e.g., experiential data or samples of input data, which are fed into the model in order to establish, tune, or modify one or more aspects of the model, e.g., the weights, biases, criteria for forming classifications or clusters, or the like. Aspects of a machine learning model may operate on an input linearly, in parallel, via a network (e.g., a neural network), or via any suitable configuration.

The execution of the machine learning model may include deployment of one or more machine learning techniques, such as linear regression, logistical regression, random forest, gradient boosted machine (GBM), deep learning, or a deep neural network. Supervised or unsupervised training may be employed. For example, supervised learning may include providing training data and labels corresponding to the training data, e.g., as ground truth. Unsupervised approaches may include clustering, classification or the like. K-means clustering or K-Nearest Neighbors may also be used, which may be supervised or unsupervised. Combinations of K-Nearest Neighbors and an unsupervised cluster technique may also be used. Any suitable type of training may be used, e.g., stochastic, gradient boosted, random seeded, recursive, epoch or batch-based, etc.

Organoids are three-dimensional, differentiated cell structures that are cultured from induced pluripotent stem cells (iPSCs) and may be used to model key features of target organs, in vitro. Organoids provide an in vitro preclinical model that combines scalability with the physiological relevance of in vivo systems. Organoids possess the human genome, meaning they recapitulate physiologically relevant circuits and are particularly valuable for modeling human-specific genomic features. This genetic fidelity enhances the translatability of findings to human diseases. As three-dimensional structures, organoids derive utility from their complex three-dimensional cytoarchitecture, which mimics the heterogeneity and layered structure of an organ, such as the human brain.

Unlike animal models, organoids are highly scalable, enabling large-scale drug screening that is not feasible with more labor-intensive methods. Moreover, physiological data from organoids are far more translatable than that derived from current preclinical models, such as conventional behavioral tests in animals, which often fail to predict human responses. Furthermore, organoids may possess alleles that are not naturally expressed in animals, such as the APOE4 allele, which is implicated in Alzheimer's disease but is not naturally expressed in rodents.

An example is a brain organoid, which may preserve critical aspects of neuronal network(s) found in intact brains, and may be used to elucidate the circuitry of the brain, model disease processes or pathologies, or test toxicity or effect(s) of drug candidates on those pathologies. It is desirable to electrically probe the interior of a brain organoid in a way that does not disturb the delicate cell connections but measures the single unit action potentials (e.g., instantaneous cell-scale electrical activity of individual neurons) and field activity (e.g., population-scale electrical activity of groups of neurons in the organoid). The systems, devices, and methods discussed herein may reliably measure the physiological activities at both single-cell and population scales, continuously, and throughout the organoid in an automated and scalable manner (e.g., measurements may efficiently be made on many organoids in a short period of time).

One current method for probing brain organoid circuitry involves the use of planar microelectrode arrays (MEAs), in which lithographically-defined conductive microelectrodes are integrated onto a planar surface, usually at the bottom of a well to which a tissue sample such as an organoid or slice is introduced. The MEA electrodes are connected to a circuit that amplifies any signal present on the electrode, such that the portions of the organoid or other tissue that are in contact with the MEA—if electrically active—may be detected by the MEA.

With this approach, the planar MEA electrodes tend to measure signals in close proximity to the electrodes, i.e. on the outer surface of the organoid. An organoid does not typically make a close connection/contact with the MEA, so to record from an intact organoid, a good deal of effort and time is expended to culture the organoid onto the surface, such that it makes more intimate contact and the resulting signal level is sufficiently robust to be measured. In practice, formation of good electrical contact is commonly prohibited by irregular organoid shapes and encapsulation by non-neuronal extracellular matrix or Matrigel, with the latter commonly used as a scaffold to promote organoid growth. Even when this process is successful, the majority of the interior of the organoid is unsampled.

More commonly than recording from the surface of intact structures, organoids are often dissociated or sliced and the resulting 2D cell layer can attach to the MEA surface. However, the main advantage of brain organoid as a more realistic model having complex, 3D cytoarchitecture is decimated. A neural circuit or microcircuit likely comprise of inter-connected neurons of diverse cell types, spanning multitude of depths within a brain organoid. Conventional MEA systems, by design, cannot sample these circuits, thus will be hopeless to produce data from the neural circuit that may have predictive power to describe the underlying disease state or drug effect.

Attempts to develop MEAs that have a or three-dimensional topography to overcome the shortcomings over current strictly planar MEA's have had little success. One issue has been developing a process to produce an electrode with sufficiently high aspect ratio to penetrate tissue effectively such that the measurement electrode is in close proximity to target neurons. It has also proven difficult to integrate multiple measurement electrodes on a single electrode “spike,” meaning that each penetrating portion of the MEA produces only a single measurement, rather than sampling along an entire trajectory of the “spike,” thus making it impossible to instrument (e.g., measure from) a complete three-dimensional sample of the organoid. Methods of fabrication have generally proven to be expensive and difficult to scale. Application also involves cumbersome and possibly destructive techniques to ensure good electrical contact by applying pressure to the organoid against the electrode array.

Another method conventionally used for recording in brain organoids is patch clamping. This method involves targeting and recording signals from one neuron at a time. Because of a lack of organization, sparsity of active neurons in current brain organoids relative to in vivo or two-dimensional cultures, this low throughput sampling can be extremely difficult to find active neurons. Even when patch clamping is successful, this method cannot simultaneously record from multiple cells that constitute a neural circuit, which may be important for understanding mechanisms related to drug efficacy and toxicity.

There is a need for improved systems, methods, and devices for improved electrical readout from organoids, including brain organisms. The embodiments disclosed herein address the above-referenced challenges by recording from inside intact organoids, capturing crucial elements of neural networks that span different regions at the spatiotemporal resolution to resolve single cell spiking activities. This capability becomes increasingly beneficial as organoids grow larger and more heterogeneous, making deep probing critical for accurate circuit-level analysis. The disclosed embodiments may perform chronic recordings of large numbers of putative single units and local field potentials.

The disclosed systems utilize high-density probes to penetrate organizes to quickly and reliably acquire data from deep within the organoids. In contrast with conventional MEA systems, the probes facilitate immediate and precise recording.

In some embodiments, a trained machine learning model may be utilized to identify effective biomarkers based on at least neural circuit behaviors and organoid-specific features. Identifying such biomarkers may allow for rapid assessment of new drug candidates. Some embodiments, as described herein, employing a trained machine learning model may utilize a classification approach, which has shown that differences in electrophysiological features are more pronounced between organoid classes (e.g., derived from different organoid maturation protocols) than within any single class. As such, utilization of the trained machine learning model may significantly increase predictive power of the model despite intrinsic variability of input data.

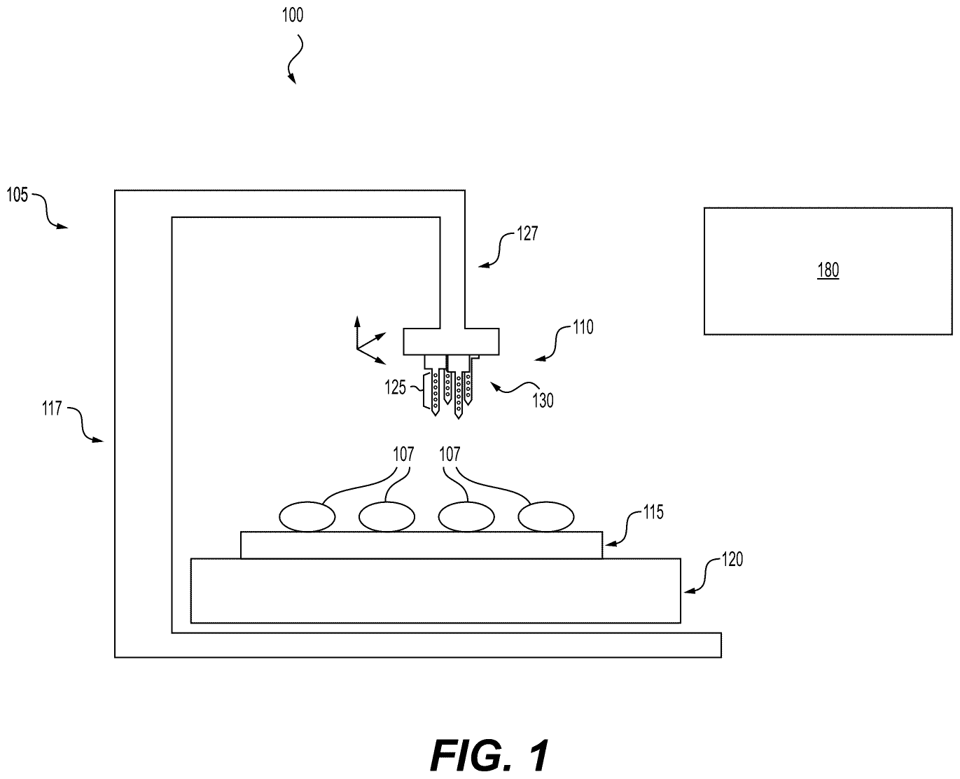

FIG. 1 depicts an organoid analysis system 100 that may be configured to conduct high-throughput recording of at least one organoid 107, such as automated recording of at least one organoid 107. As depicted in FIGS. 1-3B, organoid analysis system 100 may include an organoid analysis device 105, which may include a reader head 110, a plate 115, an alignment assembly 117 (e.g., a motorized stage 120 and/or a robotic arm 127, which may be configured to automate alignment), a camera 130, and a control and analysis unit 180 (e.g., capital equipment, such as a computer). In some examples, control and analysis unit 180 may be local to organoid analysis device 105. In alternatives, control and analysis unit 180 may be remote to organoid analysis device 105. For example, organoid analysis device 105 may communicate with control and analysis unit 180 via wired or wired (e.g., cloud-based) channels. Control and analysis unit 180 may have any of the features described with respect to FIG. 9, below.

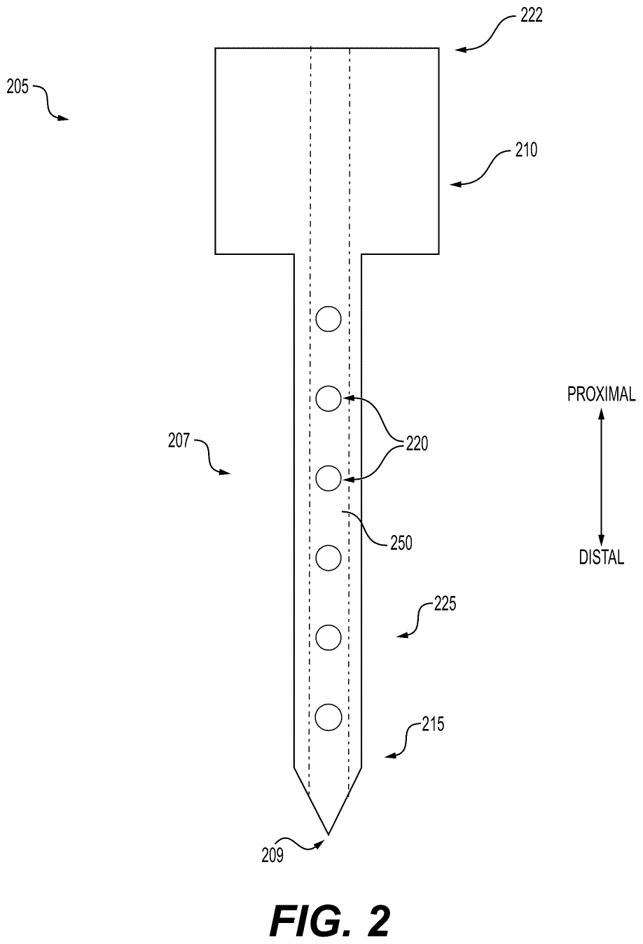

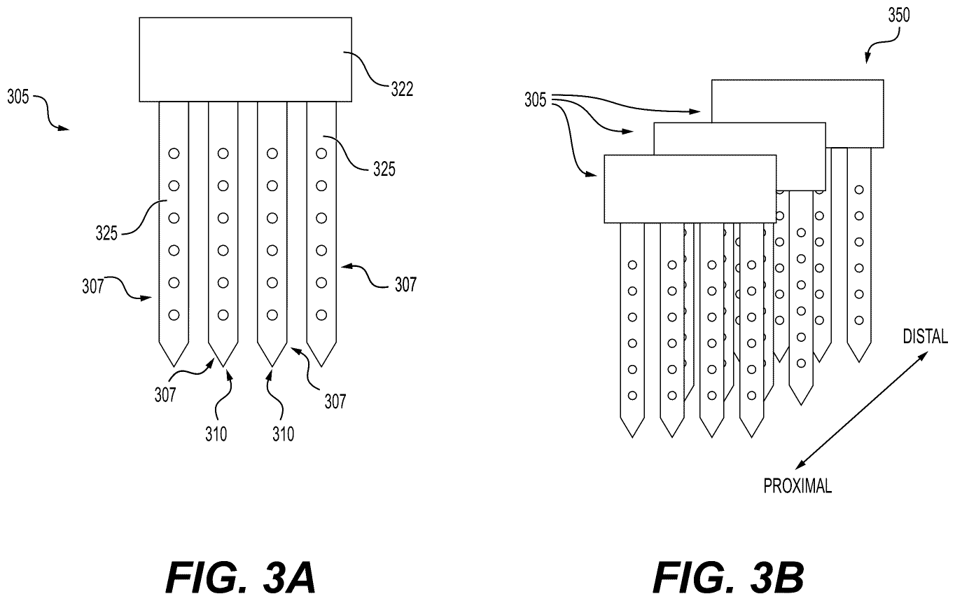

Reader head 110 may be electrically coupled to control and analysis unit 180 or other computing devices to receive data from reader head 110 and, in some examples, transmit instructions to reader head 110. Reader head 110 includes one or more needle-like structures (hereinafter referred to as “probes”) 125 that may be configured to penetrate at least one organoid 107, e.g., a brain organoid and measure electrical activity with minimal damage to the organoid. Exemplary embodiments of probe 125 are shown FIGS. 2 and 3. FIG. 2 shows an example probe 205, which may be used as probe 125. FIG. 3A shows an example probe 305, which may additionally or alternatively be used as probe 125.

As shown in FIG. 2, probe 205 may include a proximal portion 222. Proximal portion 222 may include bonding pads (not depicted and having any feature known in the art) for making electrical connection between probe 205 and reader head 110. Probe 205 may also include a distal portion 225. Distal portion 225 may include a shank 207. Shank 207 may be any suitable length, such as 6 mm, 7 mm, 8 mm, 9 mm, 10 mm, 11 mm, etc. Shank 207 may be manufactured to a given length based on the organoid categorization. For example, shank 207 may be manufactured to be 9 mm if intended for use with a brain organoid. Distal portion 225 may include micron-scale conductive recording sites 220 (e.g., electrodes), which may be configured to measure neuronal and/or other electrical activity in organoid 107. For example, recording sites 220 may measure and transmit data regarding voltages or other aspects of electrical signals of organoid 107. In examples, recording sites 220 may alternatively or additionally be stimulation sites that are configured to stimulate (e.g., deliver electrical energy to) organoid 107. For example, sites 220 may stimulate electrically active cells of organoid 107. Shank 207 may include silicon or other suitable materials. Shank 207 may include a sharp distal tip 209.

As shown in FIGS. 3A and 3B, in some embodiments, probe 305 may include multiple distal portions 325 on a plurality of shanks 307. Each of shanks 307 may have a distal portion 325. Shanks 307 and distal portions 325 may have any of the properties of shanks 207 and distal portions 225, respectively, described above. Although not labeled in FIG. 3A, distal portions 225 may include recording sites 220, described above. Each shank 307 may include a sharp distal tip 310, having any of the properties of sharp distal tip 209. The shanks 307 may optionally be parallel to one another. In at least some examples, the shanks 307 may be arranged in a single-file row, such that a plane extends through all of shanks 307. Alternatively, shanks 307 may be arranged in other patterns (e.g., multiple rows and columns). Proximal ends of shanks 307 may be coupled to a single proximal portion 322, having any of the properties of proximal portion 222. Because each shank 307 may contain an array of micron-scale conductive recording sites 220, having multiple shanks 307 on a single probe 305 structure may span a two-dimensional recording plane with shanks 307 and/or micron-scale conductive recording sites 220 strategically positioned to record from a slice of tissue.

In one embodiment, entireties or portions of probes 125, 205, 305 (e.g., shanks 207) may be comprised of a silicon substrate patterned lithographically with micron-scale conductive recording sites 220 (e.g., on distal portions 225, 325 of shanks 207, 307, respectively). The recording sites 220 may be interconnected and may allow measurements of cellular activity along a length of each shank 207, 307. Probes 125, 205, 305 may be manufactured using any suitable semiconductor fabrication techniques. Micron-scale conductive recording sites 220 may be spaced apart on shank 207, 307 in any suitable arrangement. In some embodiments, micron-scale conductive recording sites 220 may be spaced based on the intended organoid categorization. A shape of probes 125, 205, 305 and/or micron-scale conductive recording sites 220 may be optimized for the anatomy of a corresponding type(s) of organoid. For example, recording sites 220 may be arranged to have a high density of recording sites approxamtely 150-300 microns or less below the surface of organoid 107 when probe 125, 205, 305 is inserted into organoid 107, because brain organoids have a highest density of relevant structures in that region. In some embodiments, probes 125, 205, 305 may include a lumen 250 (shown in FIG. 2), which may be configured to enable injections of drugs or other compounds into organoid 107.

Furthermore, by stacking more than one probe 305 on top of each other (e.g., parallel, orthogonally, etc.), as depicted in FIG. 3B, a full three-dimensional array 350 of electrodes may be created. When array 350 of probes 305 penetrates the at least one organoid 107, the complete volume of organoid tissue may thus be sampled for electrical activity on a regular 3D grid, which may offer superior functional mapping compared to the conventional methods of surface (electrocorticography (ECoG)) or scalp (electroencephalogram (EEG)) recordings. The individual probes 305 forming the 3D array 350 may be different in designs such that the combined 3D arrangement of all recording sites 220 are configured to cover (e.g., may optimally cover) the anatomical arrangement of cells of interest within a specific type of organoid.

For purposes of immobilizing (e.g., fixing) at least one organoid 107 for penetration by reader head 110, a purpose-built culture dish (e.g., a plate 115) may be used. Plate 115 may be of the same or similar standard outer dimensions of a microwell titer plate, such that standard laboratory instruments for sample preparation, dispensing, and analysis may process the plates before and after analysis by the subject system. In some embodiments, small wells on plate 115 may help capture and immobilize organoids 107 for analysis. Plate 115 may include features such as gas or fluid pumps, microchannels or integrated heaters to maintain organoid-containing buffers and reagents at appropriate conditions to keep the organoid viable during measurements. Plate 115 may be made from transparent material such as glass, silicone-based polymers, and/or similar materials, which may be configured to allow optical imaging and/or modulation using light sources of various wavelengths. Plate 115 may have markings/markers such as fiducials (e.g., a fluorescent tag) to guide alignment of reader head 110, and/or a unique serial number that may be used to trace production history and/or specific experimental settings configured from the software.

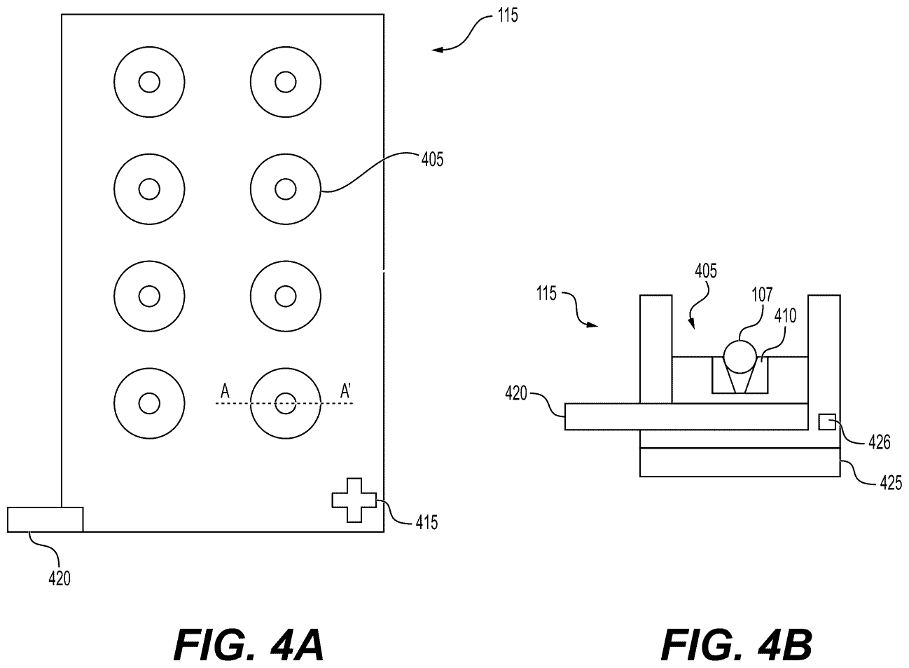

FIG. 4A depicts a top view of an exemplary plate, e.g., plate 115, and FIG. 4B depicts a cross-sectional view of an exemplary well, e.g., well 405. The cross-section view of FIG. 4B is through line A-A′ of FIG. 4A. Plate 115 may have any number of wells 405. Each well 405 may be isolated from one another (e.g., such that no cross-well fluid flow may occur to prevent contamination). Each well 405 may have a different media composition and/or interrogation compound (e.g., a drug). Each well 405 may have multiple organoid traps 410. Each organoid trap 410 may have a substantially “V”-shaped structure/shape that may be configured to immobilize organoids of varying sizes and shapes.

Plate 115 may be transparent to allow optical imaging from the bottom side. Plate 115 may have an integrated heater, e.g., heater 425, and temperature sensor 426 to control media temperature. Plate 115 may have an electrical ground 420 that is in electrical contact with a solution inside every well 405. Plate 115 may include a fiducial 415, which may be any pattern on plate 115 that has a fixed coordinate relative to the wells 405 and may recognizable by the image processing unit (not shown) to help align reader head 110 in a desired position.

As shown in FIG. 1, an alignment assembly 117, such as motorized stage 120 and/or robotic arm 127, may be used to bring reader head 110 into precise orientation and position and to penetrate, successively, each of the organoids 107 on plate 115. Positional tolerance that may approach or be within 5-10 microns in each of a Cartesian and/or spherical coordinate axes. In some embodiments, alignment assembly 117 (e.g., motorized stage 120 and/or robotic arm 127) may operate automatically. Motorized stage 120 and/or robotic arm 127 may be configured to move in an XYZ degree of freedom (e.g., along three directions that are perpendicular to one another), as well as with angular degrees of freedom around these axes. It should be noted that either of plate 115 or reader head 110 may be moved with the other held fixed to initiate precise insertion (e.g. plate 115 on motorized stage 120, or reader head 110 on robotic arm 127). In some embodiments, the position and/or orientation of both plate 115 and reader head 110 may be controlled.

High-resolution camera(s) 130 with magnifying optics may be used to image each plate 115 prior to analysis or during analysis. In alternatives, microscope(s) may be used instead of or in addition to camera(s) 130. Using image processing techniques, a center of mass of each organoid 107 on plate 115 as viewed from above by the primary camera may be determined, and a set of coordinates of those targets (e.g., organoids 107) may be stored. This information may be used to drive reader head 110 to the correct position for insertion in each organoid 107, e.g., sequentially. A secondary camera (not depicted) with line of sight perpendicular or substantially perpendicular to the insertion trajectory of probes 125, 205, 305 may be used to measure the insertion angle and distance from the probe tip(s) to the organoid surface. Additionally or alternatively, camera(s) 130 may include one or more camera(s) (or microscope(s)) that are configured to receive and/or interpret fluorescent signals from fiducials 415 on plate 115. In some examples, reader head 110 and plate 115 may both include fiducials 415, which may be fluorescent tag(s) or may be simple optical features.

Electronics and software may be used to control the mechanical mechanisms and for readout and analysis of the probe 125, 205, 305 data. Analysis software may include algorithms for characterizing organoid network activity with electrophysiological (“e-phys”) biomarkers. An e-phys biomarker is a data product that results from detailed analysis of field and single unit spiking activity. Examples include but are not limited to: gamma, alpha or sharp wave ripple (or other frequency band) power, coherence between two regions, spiking frequency, etc. E-phys biomarkers may be useful in high throughput screening of drug candidates. Further details of such analysis software is described below.

In some embodiments, the electrical readout of probe 125, 205, 305 may be used by control and analysis unit 180 during insertion in organoid 107, e.g. in closed loop configuration, to determine or select the depth (e.g., Z direction) to which probe 205, 305 should be inserted. Recording sites 220 may be in electrical communication with control and analysis unit 180, and control and analysis unit 180 may use data measured by recording sites 220 to automatically position reader head 110 and/or probes 125, 205, 305 of reader head 110. For example, an impedance measurement or neural signal processing may differentiate between different states (e.g., probe 125, 205, 305 in air; probe 125, 205, 305 in conductive solution; probe 125, 205, 305 in organoid 107 without nearby active neurons; probe 125, 205, 305 in organoid 107 with nearby active neurons, etc.).

In use, probe 125, 205, 305 may be inserted into organoid 107. Upon penetration of an organoid 107, there may be an increase in root-mean-square voltage (Vᵣₘₛ) measured by probe 125, 205, 305. For example, Vᵣₘₛ may increase by approximately two-fold upon penetration of organoid 107, which may still be within a range suitable for spike detection. In some examples, control and analysis unit 180 may analyze measurements from all or a subset of recording sites 220 (e.g., a distal subset of recording sites 220) as probe 125, 205, 305 is inserted to provide closed loop control over positioning of probe 125, 205, 305 and to halt movement (e.g., in the Z direction) of probe 125, 205, 305 before probe 125, 205, 305 is inserted too deeply. For example, movement of probe 125, 205, 305 may be halted or ceased when a threshold number of recording sites 220 measure Vᵣₘₛ below a threshold level. In some examples, a given organoid 107 may be considered to be successfully probed when at least 25% of recording sites 220 record spikes from the organoid 107.

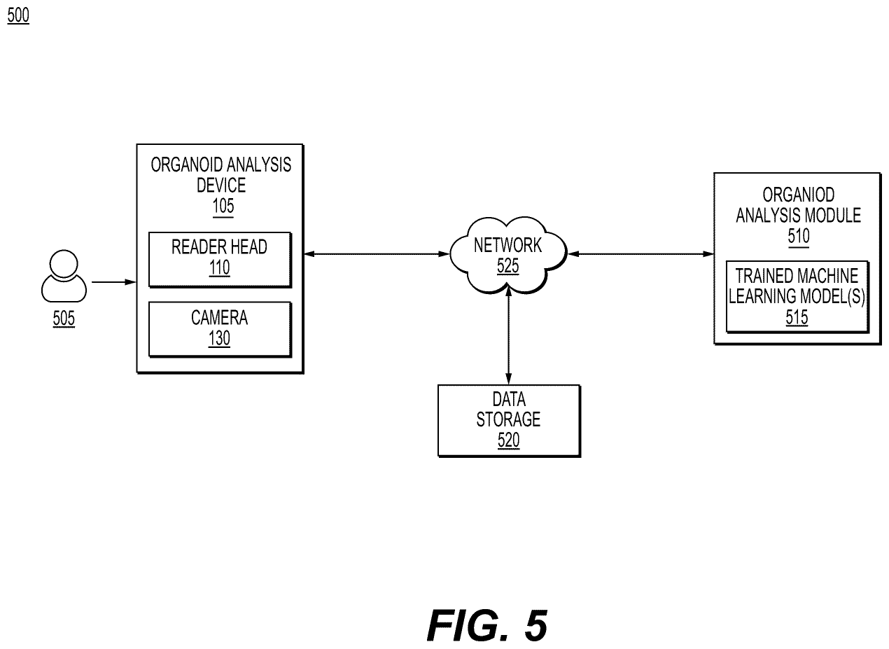

FIG. 5 depicts an exemplary environment 500 for classifying an organoid, according to one or more embodiments. For example, environment 500 may be used to classify an organoid as being a particular type of organoid. In aspects, environment 500 may classify an organoid as a dorsal forebrain organoid (dFO), a ventral forebrain organoid (vFO), or a vFO with differentiated oligodendrocytes (vFO_OL). In other aspects, environment 500 may be used to classify an organoid as being affected by a disease, such as Alzheimer's Disease (AD) or being unaffected by the disease. Environment 500 may also be utilized to screen potential treatments (e.g., drugs) in an efficient manner. The efficiency and throughput are allowed necessarily by both organoid analysis device 105 and the organoid analysis module 510. For the device, robotic control with speed and precision to move from organoid to organoid and importantly targeting each organoid with precise depth to maximize the number of sampled neurons based on closed-loop control is critical. Analysis efficiency is critically driven by trained machine learning models 515, which reduce the minimally required recording duration by orders or magnitude before predictive power of the model is compromised. The high-throughput capability, coupled with full system integration and automation, of the techniques described herein marks a substantial advancement over current technologies. The process described herein may significantly reduce time and resources required for screening large drug libraries. Unlike conventional systems that demand extensive preparation and manual intervention, the techniques described herein streamline data acquisition to ensure consistent, high-quality results. The techniques described herein enhance efficiency and reliability, minimizing human error and variability, which is crucial for accelerating the drug development process.

Environment 500 may include one or more aspects that may communicate with each other over a network 525. In some embodiments, a user 505 may interact with organoid analysis device 105 to accomplish organoid analysis. As depicted in FIG. 5, organoid analysis device 105 may interact with at least one of organoid analysis module 510 (which may be housed on control and analysis unit 180 or separately from control and analysis unit 180), a trained machine learning model(s) 515, or a data storage 520.

Organoid analysis system 100, including organoid analysis device 105, may be configured to conduct high-throughput recording of at least one organoid 107, as discussed in greater detail above. Aspects of organoid analysis device 105 (or control and analysis unit 180) may be configured to obtain data from one or more other aspects of environment 500, such as from reader head 110 or camera 130 (e.g., via one or more inputs from user 505), organoid module 510, trained machine learning model(s) 515, data storage 520, etc. Aspects of organoid analysis device 105 or control and analysis unit 180 may be configured to transmit data to one or more aspects of environment 500, such as to reader head 110, camera 130, organoid analysis module 510, trained machine learning model(s) 515, data storage 520, etc.

As discussed above, organoid analysis device 105 may include a reader head 110 and a camera 130. Reader head 110 may be configured to measure electrical activity of an organoid (e.g., electrical spike(s), local field potential (“LFP”), etc.), as discussed in greater detail herein. Reader head 110 may be configured to transmit data to one or more other aspects of environment 500, such as to organoid analysis module 510, trained machine learning model(s) 515, data storage 520, etc. In some non-limiting examples, organoid analysis module may be housed in control and analysis unit 180 (FIG. 1).

Organoid analysis module 510 may be configured to analyze at least one organoid 107, according to one or more embodiments. In some embodiments, organoid analysis module 510 may be configured to analyze at least one organoid 107 via trained machine learning model(s) 515, as discussed in more detail below. For example, organoid analysis module 510 may be configured to classify the at least one organoid 107 via trained machine learning model(s) 515, for example, by analyzing the output(s) of trained machine learning model(s) 515.

Trained machine learning model(s) 515 may be configured to classify at least one organoid 107, according to one or more embodiments. In some embodiments, a first type of trained machine learning model(s) 515 may be configured to classify the at least one organoid 107 based on spike data. Neurons fire action potentials (spikes) across a neural network. These spikes may be measured in organoids 107 by probes 125, 205, 305, discussed above. Before being utilized by machine learning model 515, electrical recordings from probes 125, 205, 305 may be processed in any suitable manner. For example, the electrical recordings may be bandpass filtered, spikes may be extracted, and metrics may be computed for each identified spike. In some examples, metrics may be computed for each spike, instead of clustering spikes by cells, in order to bypass the subjective nature of spike sorting, increase sample size, and reduce the likelihood of overfitting.

In some embodiments, a system or device other than trained machine learning model(s) 515 may be used to generate or train the machine learning model. For example, such a system may include instructions for generating the machine learning model, the training data and ground truth, or instructions for training the machine learning model. A resulting trained machine learning model may then be provided to trained machine learning model(s) 515.

Generally, a machine learning model includes a set of variables, e.g., nodes, neurons, filters, etc., that are tuned, e.g., weighted or biased, to different values via the application of training data. In supervised learning, e.g., where a ground truth is known for the training data provided, training may proceed by feeding a sample of training data into a model with variables set at initialized values, e.g., at random, based on Gaussian noise, a pre-trained model, or the like. The output may be compared with the ground truth to determine an error, which may then be back-propagated through the model to adjust the values of the variable.

Training may be conducted in any suitable manner, e.g., in batches, and may include any suitable training methodology, e.g., stochastic or non-stochastic gradient descent, gradient boosting, random forest, etc. In some embodiments, a portion of the training data may be withheld during training or used to validate the trained machine learning model, e.g., compare the output of the trained model with the ground truth for that portion of the training data to evaluate an accuracy of the trained model. The training of the machine learning model may be configured to cause the machine learning model to learn associations between training data and ground truth data, such that the trained machine learning model may be configured to determine an output illegitimate activity alert in response to the input user marker data based on the learned associations.

Each type of organoid (e.g., dFO, vFO, and vFO_OL) may have different distributions of measured spike properties. Spike properties may include, for example, amplitude, inter-spike interval, and trough-to-peak time. In other words, the probability of observing a particular value for a particular spike property may vary across organoid types. These different properties may be used to train machine learning model(s) 515 and for machine learning model(s) 515 to classify a type of organoid.

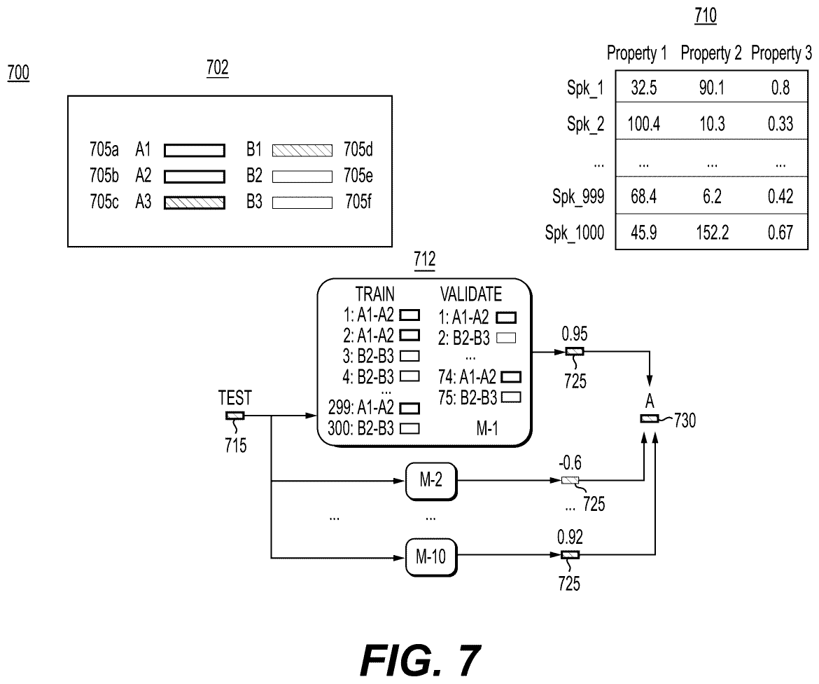

FIG. 7 depicts an exemplary schematic 700 for training and testing machine learning model(s) 515, according to one or more embodiments. For example, a bootstrapping and aggregation (Bagging) technique may be utilized to train an ensemble of machine learning models 515 (e.g., artificial neural network (ANN) models). At step 702, recordings 705a, 705b, 705c, 705d, 705e, 705f may be collected in recording sessions (e.g., from probes 125, 205, 305). These recording sessions may be bootstrapping recording sessions. Recordings 705a, 705b, and 705c may be from a first class of organoid 107, and recordings 705d, 705e, and 705f may be from a second class of organoid 107. A portion of the recording sessions from step 702 may be allocated for validating/testing machine learning model 515. For example, as depicted in FIG. 7, recordings 705c and 705d may be allocated for a model test.

At step 710, spike samples may be synthetically generated to augment the data from step 702. Synthetic spike samples may be generated for each organoid 107. For example, a portion of the spikes from different recording sessions of step 702 for the same class of organoid 107 may be used to synthetically generate new spikes for the class. For example, as shown in FIG. 7, 1000 spikes may be generated for each class. Each class of organoid may generate equally to the synthetic samples. A first portion of the synthetic samples generated in step 710 may be used for training and a second portion of the synthetic samples generated in step 710 may be used for testing/validating the machine learning model.

The data recorded in step 210 and the data synthetically generated in step 710 may include values for various properties that may distinguish classes of organoids 107 from one another. For example, as discussed above, such properties may include, for example, amplitude, inter-spike interval, and trough-to-peak time.

In step 712, model(s) 515 may be trained/validated/tested using test data 715 (e.g., the data from steps 702, 710). For example, a plurality of ANN models (e.g., ten models M-1 through M-10), as shown in FIG. 7, may be separately trained. For example, the plurality of ANN models may be trained until errors are minimized. The number of models shown in FIG. 7 is merely exemplary, and alternative numbers of machine learning models 515 may be utilized.

Models M-1 through M-10 may be tested using the data that was allocated for testing and was not sued during training. Using test data 715 as an input, each of the models M-1 through M-10 may predict a classification of the test data 715. Each of models M-1 through M-10 may output an output score 725 ranging from −1 to +1, where −1 indicates a 100% confidence in a first class, where +1 indicates a 100% confidence in a second class, and where 0 indicates pure chance. Output scores 725 may be weighted. For example, scores 725 may be weighted by the formula S_{w}=e^{c|s|}, where S_{w} is the weighted score favoring high-confidence output score 725, and c is a scaling constant. The weighted scores S_{w} may be averaged to determine a final prediction 730 of a class of organoid 107.

After testing and verification is complete, machine learning model 515 may operate in a similar manner described above (for the testing phase) for processing test data 715 in order to, in a production phase, predict a type of organoid 107. For example, a plurality of machine learning models 515 may each output a prediction score, those scores may be weighted, and then those weighted scores may be averaged in order to produce a classification of an organoid 107.

In some embodiments, in addition to the machine learning model(s) 515 described above (a first type of machine learning model 515), a second type trained machine learning model(s) 515 may be configured to classify the at least one organoid. The first type of machine learning model 515 may, in some examples, be based solely on spikes measured by recording sites 220. The second type of machine learning model 515 may also be based at least in part on local field potential (LFP). Events measured by probe 125, 205, 305 may be synchronized across different shanks 207, 307 (e.g., with a large separation distance between shanks 207, 307). In particular, these synchronized events may be measured in vFOs. These synchronized events may not only modulate spiking rate but also show LFP, which may bear similarities to in vivo phenomena, such as sharp-wave ripples (SW-R). SW-R may be important to brain functions like memory consolidation; when disrupted in animal models, the animals may show pathological conditions.

The second type of machine learning model 515 may be trained to use LFPs to classify organoid 107 (e.g., a vFO) based on the presence or absence of a condition such as AD. Machine learning model 515 may be trained by using data for organoids 107 (e.g., vFO organoids) that are known to have the condition to be classified. The training data may include LFP and spike data, such as oscillation frequency, field amplitude, synchrony power, inter-event interval, phase relationship between spike and field, or other properties. For example, the spike data may be measured during a detected synchronous event. For example, data from large amplitude, slow deflections may be utilized. These deflections may be analogous to sharp waves from the CA3 region of the hippocampus that drive ripples in the CA1 region of the hippocampus.

Combining analysis of two types of machine learning models 515—a first type of machine learning model 515 that analyzes spike data and a second type of machine learning model 515 that analyzes LFPs—may enhance accuracy in classifying organoids 107, as well as sensitivity in detecting subtle circuit-level changes related to disease models or drug effects. Bayesian Model Averaging (BMA) may be utilized to combine results from the two types of machine learning models 515. The first type of machine learning model 515 (based on spike data) may be treated as a prior in the BMA approach. Such an approach may allow consideration of the results of the second type of machine learning model 515 (using LFP data) while maintaining the predictive power of the first type of machine learning model 515 (using spike data).

For example, during training, the first type of machine learning model 515 may be trained using spike data, as described above. The second type of machine learning model 515 may be trained using LFP data from the same dataset. The accuracy of the first type of machine learning model 515 may be labeled as the prior probability P(A). The first type of machine learning model 515 (spiking model) may be used as the prior for the new LFP model. A posterior probability for the combined model may be computed using Bayes Rule: P(A|B)=P(B|A)×P(A)/P(B). P(A|B) is the posterior probability.

During a production phase, a final prediction for the ensemble of models (the one or more first type of model 515 and the one or more second type of model 515) may be a weighted average between the first and second types of machine learning models 515, with the prediction from the first type of machine learning model 515 scaled by the prior probability and the prediction from the second type of machine learning model 515 scaled by the posterior probability. This BMA approach may provide an adaptive framework in which models based on LFP data are only weighted more heavily if the second type of machine learning model 515 significantly improves prediction accuracy. The weighting of the types of machine learning model 515 may be adjusted over time, based on continued evaluation of the accuracy of the types of machine learning model 515.

Data storage 520 may obtain data from one or more aspects of organ analysis system 100, e.g., from organoid analysis device 105 (e.g., reader head 110 or camera 130), organoid analysis module 510 (e.g., trained machine learning model(s) 515), etc. Data storage 520 may transmit data to one or more aspects of organ analysis system 100, such as organoid analysis device 105 (e.g., reader head 110 or camera 130) or organoid analysis module 510 (e.g., trained machine learning model(s) 515).

One or more of the components in FIG. 5 may communicate with each other or other systems, e.g., across network 525. In some embodiments, network 525 may connect one or more components of environment 500 via a wired connection, e.g., a USB connection between organoid analysis system 100 and organoid analysis module 510. In some embodiments, network 525 may connect one or more aspects of environment 500 via an electronic network connection, for example a wide area network (WAN), a local area network (LAN), personal area network (PAN), a content delivery network (CDN), or the like. In some embodiments, the electronic network connection includes the internet, and information and data provided between various systems occurs online. “Online” may mean connecting to or accessing source data or information from a location remote from other devices or networks coupled to the Internet. Alternatively, “online” may refer to connecting or accessing an electronic network (wired or wireless) via a mobile communications network or device. The Internet is a worldwide system of computer networks—a network of networks in which a party at one computer or other device connected to the network may obtain information from any other computer and communicate with parties of other computers or devices. The most widely used part of the Internet is the World Wide Web (often-abbreviated “WWW” or called “the Web”). A “website page,” a “portal,” or the like generally encompasses a location, data store, or the like that is, for example, hosted or operated by a computer system so as to be accessible online, and that may include data configured to cause a program such as a web browser to perform operations such as send, receive, or process data, generate a visual display or an interactive interface, or the like. In any case, the connections within the environment 500 may be network, wired, any other suitable connection, or any combination thereof.

Although depicted as separate components in FIG. 5, it should be understood that a component or portion of a component in the environment 500 may, in some embodiments, be integrated with or incorporated into one or more other components. For example, organoid analysis module 510 may be integrated in organoid analysis device 105. In some embodiments, operations or aspects of one or more of the components discussed above may be distributed amongst one or more other components. Any suitable arrangement or integration of the various systems and devices of the environment 500 may be used.

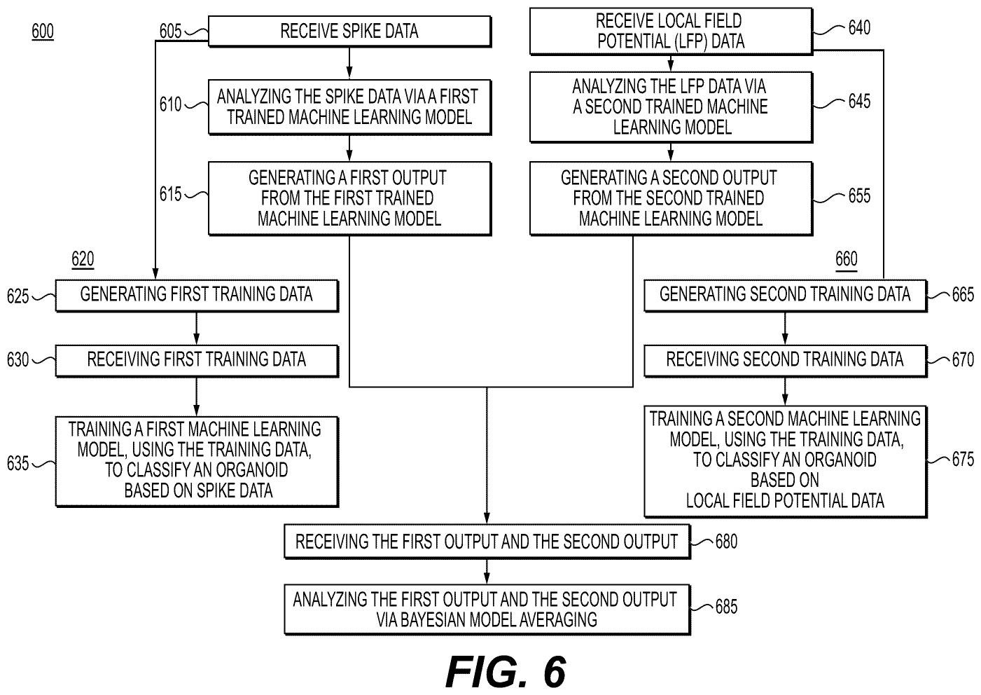

FIG. 6 depicts an exemplary method 600 for classifying an organoid, according to one or more embodiments. At step 605, spike data may be received (e.g., via organoid analysis module 510). For example, spike data may be measured (e.g., via reader head 110) and transmitted (e.g., to organoid analysis module 510).

In some embodiments, first trained machine learning model may be trained using training data via method 620. At step 625, first training data may be generated, as discussed above. The first training data may be spike data generated from one or more types of organoids (e.g., dFOs, vFOs, vFO_OLs). As discussed herein, the first training data may be measured and/or synthetic spike data for different types of organoids. The measured spike data may be processed (e.g., via bandpass filtering, extracting spikes, computing metrics for each spike, etc.) At step 630, the first training data may be received (e.g., via organoid analysis module 510).

At step 635, a first machine learning model may be trained using the first training data to generate a first trained machine learning model (e.g., a first trained machine learning model 515, which may be a spiking machine learning model 515). The first trained machine learning model may be trained to classify an organoid based on spike data.

In a production environment, at step 610, the spike data may be analyzed via a first trained machine learning model (e.g., via a first trained machine learning model 515). In some embodiments, first trained machine learning model may be trained to classify (e.g. label) at least one organoid based on spike data, as described above (e.g., a spiking machine learning model 515). At step 615, a first output (e.g., organoid classification) may be generated via the first trained machine learning model (e.g., via first trained machine learning model 515) based on the spike data.

At step 640, local field potential (“LFP”) data may be received (e.g., via organoid analysis module 510). For example, LFP data may be measured (e.g., via reader head 110) and transmitted (e.g., to organoid analysis module 510).

In some embodiments, second trained machine learning model may be trained using training data via method 660. At step 665, second training data may be generated, as discussed above. As discussed herein, the second training data may be any of the data described above, including LFP data. At step 670, the second training data may be received (e.g., via organoid analysis module 510). At step 675, a second machine learning model may be trained using the second training data to generate a second trained machine learning model (e.g., a second trained machine learning model 515). The second trained machine learning model may be trained to classify an organoid based on LFP data (and LFP machine learning model 515).

In a production environment, at step 645, LFP data may be analyzed via the second trained machine learning model (e.g., via the second trained machine learning model 515). In some embodiments, first trained machine learning model may have been trained to classify (e.g. label) at least one organoid based on LFP data. At step 655, a second output may be generated via the second trained machine learning model (e.g., via second trained machine learning model 515) based on the LFP data.

At step 680, the first output and the second output may be received (e.g., via organoid analysis module 510). At step 685, the first output and the second output may be analyzed (e.g., weighted and combined, as described above, using BMA).

Methods 600, 620, 660 may result in a highly optimized hybrid classification model. This approach may enable the model to detect subtle changes in neural network activity, particularly in response to drug candidates or to identify an organoid having a particular pathology.

While traditional single-unit electrophysiology offers a necessary baseline understanding, it lacks the throughput and sensitivity to detect subtle patterns crucial for drug screening. The techniques described herein reduce high-dimensional data into physiologically relevant features, derived from spikes or local field potentials.

The high-throughput capability, coupled with full system integration and automation, of the techniques described herein marks a substantial advancement over current technologies. The process described herein—from spike detection and feature extraction to classification—results in significantly reduced time and resources required for screening large drug libraries. Unlike conventional systems that demand extensive preparation and manual intervention, the techniques described herein streamline data acquisition to ensure consistent, high-quality results. The techniques described herein enhance efficiency and reliability, minimizing human error and variability, which is crucial for accelerating the drug development process.

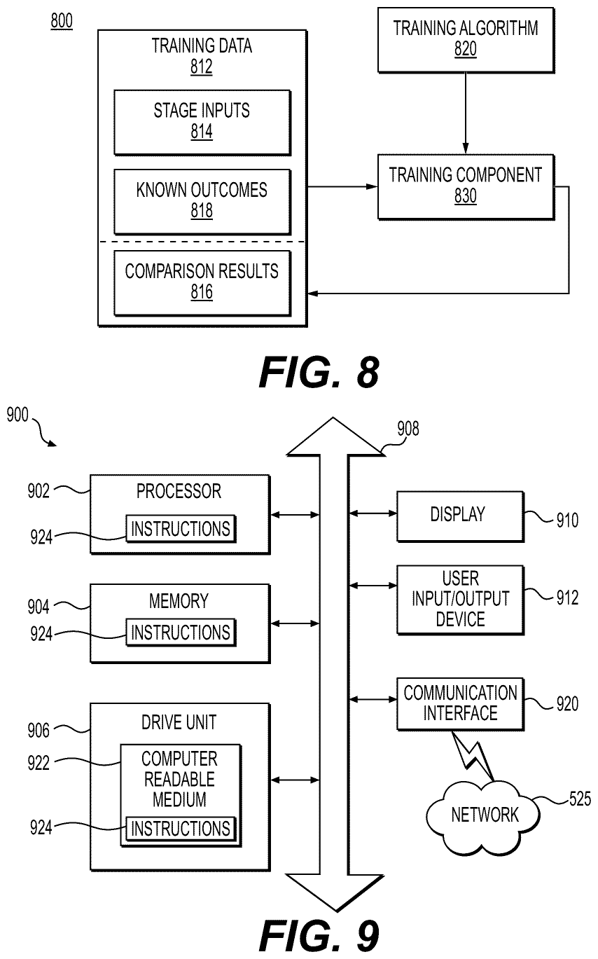

One or more implementations disclosed herein include or are implemented using a machine learning model (e.g., trained machine learning model(s) 515) are implemented using a machine learning model or are used to train the machine learning model. A given machine learning model may be trained using the training flow chart 800 of FIG. 8. The training data 812 may include one or more of stage inputs 814 and the known outcomes 818 related to the machine learning model to be trained. The stage inputs 814 are from any applicable source including text, visual representations, data, values, comparisons, and stage outputs, e.g., one or more outputs from one or more steps from FIGS. 6-7. The known outcomes 818 are included for the machine learning models generated based on supervised or semi-supervised training. An unsupervised machine learning model is not trained using the known outcomes 818. The known outcomes 818 includes known or desired outputs for future inputs similar to or in the same category as the stage inputs 814 that do not have corresponding known outputs.

The training data 812 and a training algorithm 820, e.g., one or more of the modules implemented using the machine learning model or are used to train the machine learning model, is provided to a training component 830 that applies the training data 812 to the training algorithm 820 to generate the machine learning model. According to an implementation, the training component 830 is provided comparison results 816 that compare a previous output of the corresponding machine learning model to apply the previous result to re-train the machine learning model. The comparison results 816 are used by the training component 830 to update the corresponding machine learning model. The training algorithm 820 utilizes machine learning networks or models including, but not limited to a deep learning network such as a transformer, Deep Neural Networks (DNN), Convolutional Neural Networks (CNN), Fully Convolutional Networks (FCN) and Recurrent Neural Networks (RCN), probabilistic models such as Bayesian Networks and Graphical Models, classifiers such as K-Nearest Neighbors, or discriminative models such as Decision Forests and maximum margin methods, the model specifically discussed herein, or the like.

The machine learning model used herein is trained or used by adjusting one or more weights or one or more layers of the machine learning model. For example, during training, a given weight is adjusted (e.g., increased, decreased, removed) based on training data or input data. Similarly, a layer is updated, added, or removed based on training data/and or input data. The resulting outputs are adjusted based on the adjusted weights or layers.

FIG. 9 depicts a simplified functional block diagram of a computer 900 that may be configured as a device for executing the methods disclosed here, according to exemplary embodiments of the present disclosure. For example, the computer 900 may be configured as a system according to exemplary embodiments of this disclosure. In various embodiments, any of the systems herein may be a computer 00 including, for example, a data communication interface 920 for packet data communication. The computer 900 also may include a central processing unit (CPU) 902, in the form of one or more processors, for executing program instructions. The computer 900 may include an internal communication bus 908, and a storage unit 906 (such as ROM, HDD, SDD, etc.) that may store data on a computer readable medium 922, although the computer 900 may receive programming and data via network communications. The computer 900 may also have a memory 904 (such as RAM) storing instructions 924 for executing techniques presented herein, although the instructions 924 may be stored temporarily or permanently within other modules of computer 900 (e.g., processor 902 or computer readable medium 922). The computer 900 also may include input and output ports 912 or a display 910 to connect with input and output devices such as keyboards, mice, touchscreens, monitors, displays, etc. The various system functions may be implemented in a distributed fashion on a number of similar platforms, to distribute the processing load. Alternatively, the systems may be implemented by appropriate programming of one computer hardware platform.

Program aspects of the technology may be thought of as “products” or “articles of manufacture” typically in the form of executable code or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer of the mobile communication network into the computer platform of a server or from a server to the mobile device. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

It should be appreciated that in the above description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

Thus, while certain embodiments have been described, those skilled in the art will recognize that other and further modifications may be made thereto without departing from the spirit of the invention, and it is intended to claim all such changes and modifications as falling within the scope of the invention. For example, functionality may be added or deleted from the block diagrams and operations may be interchanged among functional blocks. Steps may be added or deleted to methods described within the scope of the present invention. The above disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description. While various implementations of the disclosure have been described, it will be apparent to those of ordinary skill in the art that many more implementations are possible within the scope of the disclosure. Accordingly, the disclosure is not to be restricted except in light of the attached claims and their equivalents.

What is claimed is:

1. A device for organoid analysis, the device including:
a reader head having a plurality of probes, wherein each of the plurality of probes includes a shank with a plurality of recording or stimulation sites; and
a plate having a plurality of wells, wherein each well of the plurality of wells is configured to receive at least one organoid;
wherein the reader head is configured to automatically probe the at least one organoid and to measure a property of the at least one organoid or to stimulate electrically active cells using the plurality of recording or stimulation sites of the plurality of probes.

2. The device of claim 1, further comprising a robotic arm coupled to the reader head, wherein the robotic arm is configured to automatically position the reader head relative to a well of the plurality of wells.

3. The device of claim 1, further comprising a motorized stage configured to automatically position the plate.

4. The device of claim 1, wherein the shank includes a sharp tip.

5. The device of claim 1, wherein each well of the plurality of wells includes at least one organoid trap, and wherein the at least one organoid trap has a substantially “V”-shaped shape to immobilize an organoid of the at least one organoid received in the organoid trap.

6. The device of claim 1, wherein the device further includes a camera.

7. The device of claim 6, wherein the plate includes at least one fiducial, and wherein the camera is configured to interpret the at least one fiducial, and wherein the reader head is automatically aligned with a well of the plurality of wells based on interpretation of the at least one fiducial.

8. The device of claim 1, wherein the plurality of recording or stimulation sites are lithographically patterned on the shank.

9. The device of claim 1, wherein the plurality of recording or stimulation sites are in electrical communication with a control and analysis unit, and wherein the control and analysis unit is configured to automatically position the plurality of probes based on data measured by the plurality of recording sites.

10. The device of claim 9, wherein the control and analysis unit is configured to cease advancement of the plurality of probes based on the data measured by the plurality of recording sites.

11. The device of claim 9, wherein the control and analysis unit is configured to automatically select a depth at which to position the plurality of probes within an organoid of the at least one organoid based on the data measured by the plurality of recording sites.

12. A device for organoid analysis, the device including:
a plurality of probes, wherein each of the plurality of probes includes a shank with a plurality of recording or stimulation sites;
a camera; and
a plate having a plurality of wells, wherein each well of the plurality of wells is configured to receive at least one organoid, and wherein the plate includes at least one fiducial;
wherein the camera is configured to interpret the at least one fiducial, and wherein the plurality of probes aligned with a well of the plurality of wells based on interpretation of the at least one fiducial.

13. The device of claim 12, further comprising a robotic arm coupled to plurality of probes, wherein the robotic arm is configured to automatically position the plurality of probes relative to the well of the plurality of wells.

14. The device of claim 12, wherein each well of the plurality of wells includes at least one organoid trap, and wherein the at least one organoid trap has a substantially “V”-shaped shape to immobilize an organoid of the at least one organoid received in the organoid trap.

15. The device of claim 12, wherein the plurality of recording or stimulation sites are in electrical communication with a control and analysis unit, and wherein the control and analysis unit is configured to automatically position the plurality of probes based on data measured by the plurality of recording sites.

16. The device of claim 15, wherein the control and analysis unit is configured to cease advancement of the plurality of probes based on the data measured by the plurality of recording sites.

17. The device of claim 15, wherein the control and analysis unit is configured to automatically select a depth at which to position the plurality of probes within an organoid of the at least one organoid based on the data measured by the plurality of recording sites.

18. A system for organoid analysis, the system including:
an organoid probing device having:
at least one probe including a shank with a plurality of recording sites; and
a plate having a plurality of wells, wherein each well of the plurality of wells is configured to receive at least one organoid; and
a control and analysis unit configured to receive data from the plurality of recording sites and to automatically position the at least one probe based on the data.

19. The system of claim 18, wherein the control and analysis unit is configured to cease advancement of the at least one probe based on the data from the plurality of recording sites.

20. The system of claim 18, wherein each well of the plurality of wells includes at least one organoid trap, and wherein the at least one organoid trap has a substantially “V”-shaped shape to immobilize an organoid of the at least one organoid received in the organoid trap. each of the wells includes a V-shaped.
